(19) United States
(12) Patent Application Publication
(10) Pub. No.: US 2022/0296413 A1
(43) Pub. Date: Sep. 22, 2022

(54) Targeted Temperature Management Pad Design

(71) Applicant: C. R. Bard, Inc., Franklin Lakes, NJ (US)

(72) Inventor: Jill Walthall Jones, Avondale Estates, GA (US)

(21) Appl. No.: 17/694,416

(22) Filed: Mar. 14, 2022

Related U.S. Application Data
(60) Provisional application No. 63/162,916, filed on Mar. 18, 2021.

(51) Int. Cl. A61F 7/02

(52) U.S. Cl. CPC A61F 7/02; A61F 2007/0054

(57) ABSTRACT

Disclosed herein is a system, apparatus and method directed to an orientable and resizable targeted temperature management (TTM) pad. The system, apparatus and method pertain to a medical pad for exchanging thermal energy between a TTM fluid and a patient. The pad can include a fluid containing layer for containing the TTM fluid, a patient contact surface to facilitate thermal energy exchange with the patient, a central trunk structure, and at least two wings branching from the central trunk structure. The fluid containing layer can be configured for circulating the TTM fluid within the fluid containing layer. The patient contact surface can be disposed at least on part of the central trunk structure. The at least two wings can be configured to be wrapped around a portion of a body of the patient. The central trunk structure may be positioned beneath the patient. The wings may be trimmed, reducing the pad's size.

### PRIORITY

This application claims the benefit of priority to U.S. Provisional Application No. 63/162,916, filed Mar. 18, 2021, which is incorporated by reference in its entirety into this application.

### SUMMARY

Briefly summarized, embodiments disclosed herein are directed to systems, methods and apparatuses for an easily orientable, resizable, and wearable medical pad for Targeted Temperature Management (TTM) procedures, that is, for cooling or heating a patient to provide medical benefits, such as neuroprotection following a stroke or surgery.

One problem that often arises with targeted temperature management (TTM) systems is the difficulty for clinical practitioners to place the TTM pads on a patient expeditiously and properly. In particular, properly placing a TTM pad may not be based on aligning the pad with obvious axes of the patient's body, and thus it may be difficult to orient the pads correctly. In the case of TTM pads designed to cover the patient's torso, two independent pads are required, posing additional concerns, such as orienting both pads, and aligning the two pads parallel to each other. Likewise, it may be difficult for a clinician to wrap the pad about the patient's body, as the clinician may need to turn the patient on his or her side while placing the pads.

A second problem is the need for TTM medical pads to accommodate different patient sizes effectively and comfortably, and the further clinical confusion and frustration this may cause. An ill-fitting medical pad may hinder effective transmission of thermal energy between the pad and the patient, and may also give rise to patient discomfort. For example, if the TTM pad is oversized, covering too much of a patient's body surface, the patient may be heated or cooled too strongly by the TTM pad. In another example, an undersized TTM pad may not heat or cool the patient sufficiently. As a result, conventionally, numerous sizes are available, but selecting the correct size may be difficult and complex, as well as requiring multiple sizes to be maintained in storage. Moreover, there exists a risk of selecting the wrong size pad and making multiple kit components unusable, resulting in waste of a valuable product and frustration on the part of users due to incorrect pad sizing.

Disclosed herein is a medical pad for exchanging thermal energy between a TTM fluid and a patient. The pad comprises a fluid containing layer for containing the TTM fluid, a patient contact surface to facilitate thermal energy exchange with the patient, a central trunk structure, and at least two wings branching from the central trunk structure. The fluid containing layer is configured for circulating the TTM fluid within the fluid containing layer. The patient contact surface is disposed at least on part of the central trunk structure. The at least two wings are configured to be wrapped around a portion of a body of the patient.

In some embodiments, a respective wing of the at least two wings is further configured to be trimmed, thereby reducing a size of the medical pad.

In some embodiments, the patient contact surface is substantially aligned over the fluid containing layer, thereby facilitating the thermal energy exchange with the TTM fluid circulating within the fluid containing layer. The respective wing is configured to be trimmed in a respective edge section of the respective wing. The fluid containing layer does not extend within the respective edge section. The reducing the size of the medical pad does not reduce an area of the patient contact surface.

In some embodiments, the medical pad further comprises a conformable, thermally conductive hydrogel layer. The hydrogel layer extends to the respective edge section of the respective wing. The hydrogel layer is configured to be trimmed together with the respective wing.

In some embodiments, the patient contact surface is substantially aligned over the fluid containing layer, thereby facilitating the thermal energy exchange with the TTM fluid circulating within the fluid containing layer. The patient contact surface is further disposed at least on a respective section of the respective wing. The fluid containing layer is disposed at least partly within the respective section of the respective wing. The respective wing is configured to be trimmed in the respective section. The reducing the size of the medical pad reduces an area of the patient contact surface.

In some embodiments, the at least two wings comprise a plurality of pairs of wings. Each pair of wings comprises two wings on opposite sides of the central trunk structure. Each pair of wings is configured to be wrapped around the portion of the body of the patient.

In some embodiments, the portion of the body of the patient comprises a leg of the patient.

In some embodiments, the portion of the body of the patient comprises a torso of the patient.

In some embodiments, the central trunk structure is configured to be positioned beneath the patient. The at least two wings are configured to be wrapped around to a front of the portion of the body of the patient.

In some embodiments, the at least two wings are configured to be fastened together via a strap.

In some embodiments, the strap is connected to the at least two wings via hook and loop fasteners.

In some embodiments, the strap is connected to the at least two wings via a snap, button, zipper, or clasp.

In some embodiments, the strap is sewn, attached, or fixed to a first wing of the at least two wings.

Also disclosed herein is a universal medical pad for exchanging thermal energy between a targeted temperature management (TTM) fluid and a patient. The universal medical pad comprises a fluid containing layer for containing the TTM fluid and a patient contact surface to facilitate thermal energy exchange with the patient. The fluid containing layer is configured for circulating the TTM fluid within the fluid containing layer. The universal medical pad is configured to be trimmed, thereby reducing a size of the universal medical pad.

Also disclosed herein is a method of providing a targeted temperature management (TTM) therapy to a patient. The method includes, in some embodiments, providing a TTM system comprising a TTM module configured to provide a TTM fluid, a thermal pad configured to receive the TTM fluid from the TTM module to facilitate thermal energy transfer between the TTM fluid and a patient, and a fluid delivery line (FDL) extending between the TTM module and the thermal pad. The FDL is configured to provide TTM fluid flow between the TTM module and the thermal pad. The thermal pad comprises a patient contact surface to facilitate thermal energy exchange with the patient, a central trunk structure, and at least two wings branching from the central trunk structure. The patient contact surface is disposed at least on part of the central trunk structure. The method further includes applying the pad to the patient. Applying the pad comprises wrapping the at least two wings around a portion of a body of the patient. The method further includes delivering TTM fluid from the TTM module to the thermal pad.

These and other features of the concepts provided herein will become more apparent to those of skill in the art in view of the accompanying drawings and following description, which disclose particular embodiments of such concepts in greater detail.

### BRIEF DESCRIPTION OF THE DRAWINGS

Embodiments of the disclosure are illustrated by way of example and not by way of limitation in the figures of the accompanying drawings, in which like references indicate similar elements and in which:

FIG. 1 illustrates a TTM system using medical pads for heating and/or cooling a patient, according to some embodiments;

FIG. 2 illustrates TTM medical pads being placed on a patient, according to some embodiments;

FIG. 3 illustrates a structure of an exemplary medical pad, according to some embodiments;

FIG. 4 shows a patient with an oversized TTM medical pad;

FIG. 5A illustrates an exemplary orientable and resizable medical pad for a torso, according to some embodiments;

FIG. 5B illustrates an exemplary orientable and resizable medical pad for legs, according to some embodiments;

FIG. 5C illustrates an exemplary orientable and resizable universal medical pad, according to some embodiments;

FIGS. 6A-6B illustrates placement of an exemplary orientable and resizable medical pad on a patient, according to some embodiments;

FIG. 7 illustrates trimming of an exemplary orientable and resizable medical pad, according to some embodiments;

FIG. 8A provides an exploded perspective view of a TTM fluid filter, according to some embodiments;

FIG. 8B provides a cross-sectional side view of the filter of FIG. 8A, according to some embodiments;

FIG. 8C provides a side cross-sectional view of the thermal contact pad of FIG. 3 incorporating the filter of FIG. 8A, according to some embodiments; and

FIG. 9 illustrates a method of using an orientable and resizable TTM medical pad, according to some embodiments.

### DETAILED DESCRIPTION

Before some particular embodiments are disclosed in greater detail, it should be understood that the particular embodiments disclosed herein do not limit the scope of the concepts provided herein. It should also be understood that a particular embodiment disclosed herein can have features that can be readily separated from the particular embodiment and optionally combined with or substituted for features of any of a number of other embodiments disclosed herein.

Regarding terms used herein, it should also be understood the terms are for the purpose of describing some particular embodiments, and the terms do not limit the scope of the concepts provided herein. Ordinal numbers (e.g., first, second, third, etc.) are generally used to distinguish or identify different features or steps in a group of features or steps, and do not supply a serial or numerical limitation. For example, “first,” “second,” and “third” features or steps need not necessarily appear in that order, and the particular embodiments including such features or steps need not necessarily be limited to the three features or steps. Labels such as “left,” “right,” “top,” “bottom,” “front,” “back,” and the like are used for convenience and are not intended to imply, for example, any particular fixed location, orientation, or direction. Instead, such labels are used to reflect, for example, relative location, orientation, or directions. Singular forms of “a,” “an,” and “the” include plural references unless the context clearly dictates otherwise.

With respect to “proximal,” a “proximal portion” or a “proximal end portion” of, for example, a probe disclosed herein includes a portion of the probe intended to be near a clinician when the probe is used on a patient. Likewise, a “proximal length” of, for example, the probe includes a length of the probe intended to be near the clinician when the probe is used on the patient. A “proximal end” of, for example, the probe includes an end of the probe intended to be near the clinician when the probe is used on the patient. The proximal portion, the proximal end portion, or the proximal length of the probe can include the proximal end of the probe; however, the proximal portion, the proximal end portion, or the proximal length of the probe need not include the proximal end of the probe. That is, unless context suggests otherwise, the proximal portion, the proximal end portion, or the proximal length of the probe is not a terminal portion or terminal length of the probe.

With respect to “distal,” a “distal portion” or a “distal end portion” of, for example, a probe disclosed herein includes a portion of the probe intended to be near or in a patient when the probe is used on the patient. Likewise, a “distal length” of, for example, the probe includes a length of the probe intended to be near or in the patient when the probe is used on the patient. A “distal end” of, for example, the probe includes an end of the probe intended to be near or in the patient when the probe is used on the patient. The distal portion, the distal end portion, or the distal length of the probe can include the distal end of the probe; however, the distal portion, the distal end portion, or the distal length of the probe need not include the distal end of the probe. That is, unless context suggests otherwise, the distal portion, the distal end portion, or the distal length of the probe is not a terminal portion or terminal length of the probe.

The term “logic” may be representative of hardware, firmware or software that is configured to perform one or more functions. As hardware, the term logic may refer to or include circuitry having data processing and/or storage functionality. Examples of such circuitry may include, but are not limited or restricted to a hardware processor (e.g., microprocessor, one or more processor cores, a digital signal processor, a programmable gate array, a microcontroller, an application specific integrated circuit “ASIC”, etc.), a semiconductor memory, or combinatorial elements.

Additionally, or in the alternative, the term logic may refer to or include software such as one or more processes, one or more instances, Application Programming Interface(s) (API), subroutine(s), function(s), applet(s), servlet(s), routine(s), source code, object code, shared library/dynamic link library (dll), or even one or more instructions. This software may be stored in any type of a suitable non-transitory storage medium, or transitory storage medium (e.g., electrical, optical, acoustical or other form of propagated signals such as carrier waves, infrared signals, or digital signals). Examples of a non-transitory storage medium may include, but are not limited or restricted to a programmable circuit; non-persistent storage such as volatile memory (e.g., any type of random access memory “RAM”); or persistent storage such as non-volatile memory (e.g., read-only memory “ROM”, power-backed RAM, flash memory, phase-change memory, etc.), a solid-state drive, hard disk drive, an optical disc drive, or a portable memory device. As firmware, the logic may be stored in persistent storage.

One problem that often arises with targeted temperature management (TTM) systems is that it is difficult for clinical practitioners to place the TTM pads on a patient properly and expeditiously. In particular, it may be confusing or complex to orient the pad 120 correctly, and to wrap the pad 120 about the patient's body. The disclosed embodiments of TTM medical pads and methods can address this problem by providing a simple, adjustable, universally-accessible approach to placing and wrapping the TTM medical pads about a patient.

In addition, the need for TTM medical pads to accommodate different patient sizes comfortably and effectively can cause further clinical confusion and frustration. An ill-fitting medical pad may hinder effective transmission of thermal energy between the pad and the patient, and may also give rise to patient discomfort. For example, if the TTM pad is oversized, covering too much of a patient's body surface, the patient may be heated or cooled too strongly by the TTM pad. In another example, an undersized TTM pad may not heat or cool the patient sufficiently. Therefore, numerous sizes of conventional TTM pads are typically available to fit patients of different sizes. However, selecting the correct size may be difficult and complex, and hospitals or other facilities must devote valuable storage space to stocking numerous TTM pad sizes. Moreover, there exists a risk of selecting the wrong size pad and making multiple kit components unusable, resulting in waste of a valuable product and frustration on the part of users due to incorrect pad sizing. The disclosed embodiments of TTM medical pads and methods can address this problem by adjusting the patient contact area of the thermal pad to better accommodate different patient sizes.

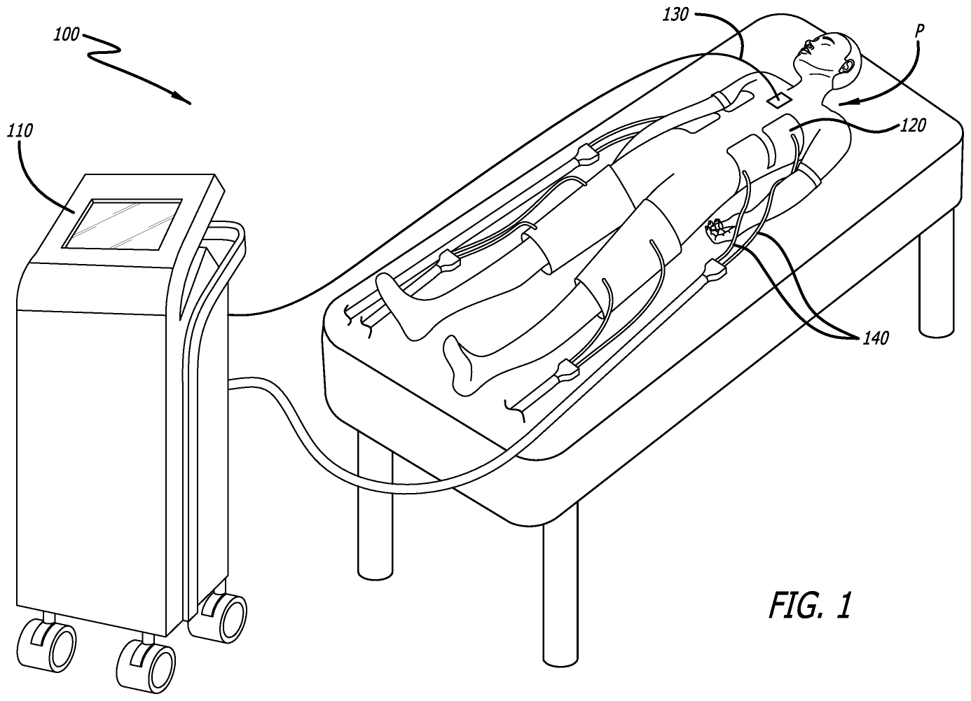

Reference is now made to FIG. 1, which illustrates a TTM system 100 using medical pads 120 for heating and/or cooling a patient P, according to some embodiments. The illustrated patient temperature control system 100 is a thermoregulatory system and apparatus that monitors and controls patient temperature within a range of 32° C. to 38.5° C. (89.6° F. to 101.3° F.). TTM system 100 is selectively interconnected to one or more medical contact pads 120 for exchanging thermal energy with patient P, and can also include a circulating pump for drawing temperature-controlled fluid (e.g., water or a gas) through pads 120 under negative pressure.

In some embodiments, TTM system 100 can include a control module 110, one or more disposable medical contact pads 120, a remote display in control module 110, a patient temperature probe 130, one or more fluid circulation lines 140, such as inlet and outlet lines to and from pads 120, and any additional accessories. In a typical embodiment, there may be two pads 120 placed on the patient's upper body as shown, and two on the patient's lower body. The TTM system 100 uses negative pressure to draw temperature-controlled fluid, such as water ranging between 4° C. and 42° C. (39.2° F. and 107.6° F.), through the pads 120 at approximately 0.7 liters per minute per pad. This results in heat exchange between the circulating fluid and the patient P. The patient temperature probe 130 is connected to the control module 110, and provides patient temperature feedback information to an internal control algorithm of control module 110. Based on such an internal control algorithm, control module 110 can increase or decrease the circulating water temperature so as to heat or cool patient P to a target patient temperature, which can be set by the clinician.

Fluid circulation lines 140 may include opposing tubing assemblies for interconnection to outlet and inlet ports of the circulating pump, with pads 120 fluidly interconnectable by means of opposing pad manifolds. FIG. 1 also illustrates the interconnection of one or more external patient temperature sensors 130 with a signal conditioning interface of control module 110. The temperature information received from external temperature sensors 130 may be utilized at a processor of control module 110 to determine the amount and rate of thermal exchange to be affected by system 100 in relation to the preset or user-defined patient target temperature. Accordingly, the processor may provide appropriate control drive signals to a heater, radiator/fan and/or auxiliary pump of TTM system 100. In an embodiment, the circulating pump, heater, radiator/fan, and/or auxiliary pump may be housed within control module 110.

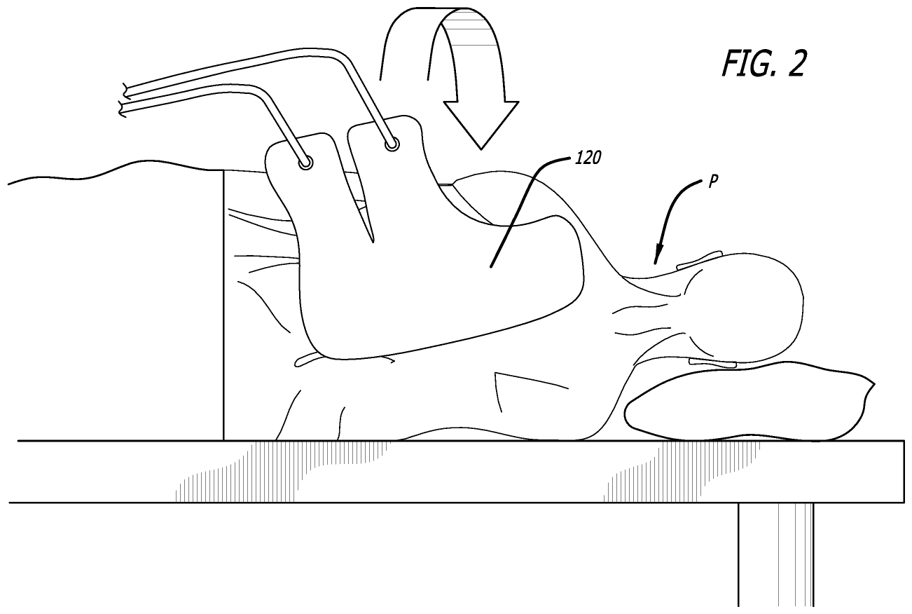

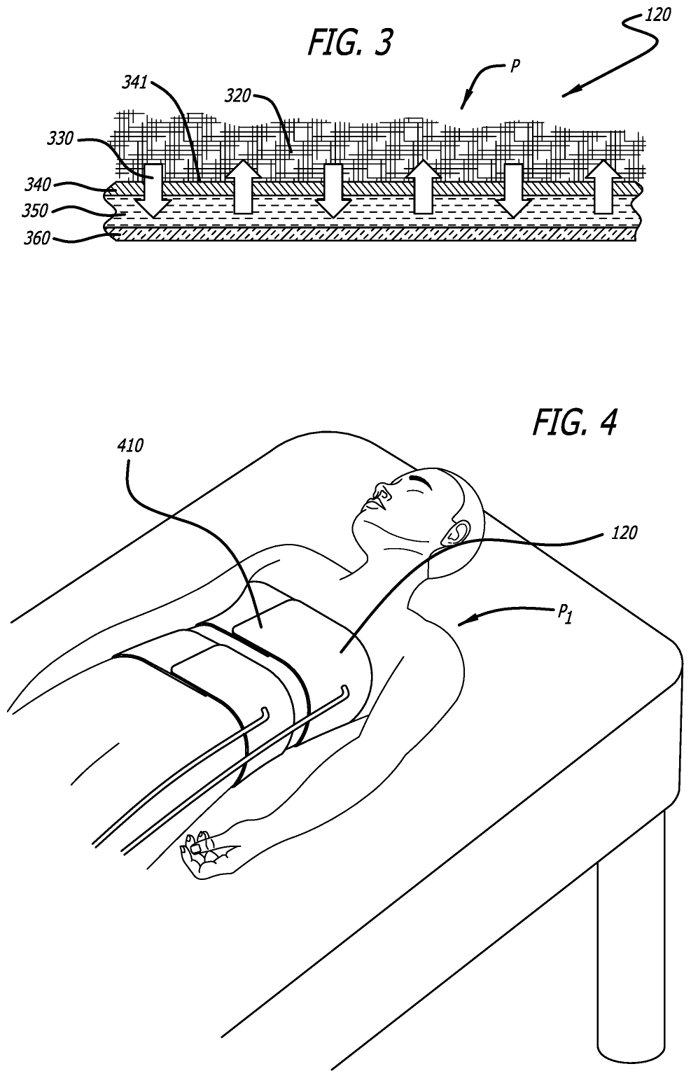

FIG. 2 illustrates a TTM medical pad 120 being placed on a patient P, according to some embodiments. Pad 120, and particularly an inner layer of pad 120 containing biocompatible hydrogel, can conform to the patient's skin, and thereby provide good thermal contact with patient P. The medical pad 120 can include several layers: an inner biocompatible hydrogel layer that adheres and conforms to the patient P, a fluid containing layer, one or more thin film layers which serve as a fluid barrier, and an outer insulating layer which prevents heat transfer to the environment (see FIG. 3). The hydrogel layer can have sufficient adhesive strength to hold pads 120 in place on patient P during the TTM therapy, yet not cause tissue damage when subsequently removed.

The pads may be available in extra-small, small, medium, and large sizes, as well as a universal pad. The clinician can determine the style, size, and number of pads 120 to be applied to patient P based on the patient procedure, application, or the available body surface area on patient P. For example, the clinician may place two pads 120 on the patient's upper body, such as on the patient's back and torso as illustrated in FIG. 2, and two pads on the patient's lower body, for example wrapped around the patient's thighs. The medical pads 120 will provide the best performance when the maximum number and correct size are used.

Due to the negative fluid pressure applied by system 100, significant fluid leakage will not occur, even if pads 120 are damaged or broken while fluid is flowing. Accordingly, pads 120 can be applied to the patient while fluid is already flowing through the pads. Depending on the objective of the treatment and the patient's level of consciousness or awareness, pads 120 may be pre-warmed or pre-cooled prior to placement.

In order to place TTM pad 120, a clinician will first align the top of a first upper body pad 120 with axilla of the patient's outstretched arm. The clinician will then place the long side of pad 120 along the side of the patient's spine. Next, the clinician can wrap pad 120 from back to front as illustrated, ensuring that the pad's fluid inlet and outlet lines are lying anteriorly. For the lower body, the clinician can align the first lower body pad's lines with the knee and point downward. The clinician will wrap the long end of the first lower body pad laterally, and overlap medially if needed.

The clinician may then turn the patient P and place a second upper body pad on the patient's other side, leaving a space along the patient's spine. Next, the clinician can wrap a second lower body pad around the patient's other leg, ensuring that the shorter edge is placed medially and the longer side is wrapped laterally. Finally, if additional surface coverage is needed, the clinician can optionally place a universal medical pad on the patient's abdomen.

The medical pads 120 have inlet and outlet lines for the fluid flow, referred to herein as pad lines (see FIG. 1). These lines are connected to the pads 120 by means of a pad manifold. In particular, a Y-shaped fluid delivery line (FDL) contains one-way valves that connect to pad line connectors (e.g., a total of six connectors). Each side of the fluid delivery line can be placed by the patient's feet or along the patient's lower legs. The connectors can accommodate a full set of four pads 120 plus a maximum of two optional universal medical pads for larger patients. While holding the pad line tubing, the clinician can insert a pad line connector into the pad fluid delivery line manifold. For example, the clinician can push a respective connector toward the manifold to release associated catches, and then pull apart. Subsequently, the clinician can disconnect the lines, e.g., by squeezing wings on the connector together.

The method of placing pad 120 outlined in the example of FIG. 2 may be difficult for clinical practitioners to follow. In particular, it may be confusing or complex to orient the pad 120 correctly, and to wrap the pad 120 about the patient's body. The disclosed embodiments of TTM medical pads and methods can address this problem by providing a simple, adjustable, universally-accessible approach to placing and wrapping the TTM medical pads about a patient.

FIG. 3 illustrates a structure of an exemplary medical pad, according to some embodiments. TTM medical pad 120 comprises inner biocompatible hydrogel layer 340, which is a conformable, thermally conductive layer that can adjoin and conform to patient's skin 320. Further, the pad 120 may include an adhesive layer 341 disposed on the skin contacting side of the hydrogel layer 340 for adhering the pad 120 to the patient's skin 320. While not shown, a removable release liner may be provided over the adhesive surface 341 to protect the adhesive surface 341 from contamination while the pad 120 is not in use.

Pad 120 additionally comprises fluid containing layer 350 and insulation layer 360 for preventing loss of thermal energy to the environment. The fluid containing layer 350 can be defined between one or more film layers and/or insulation layer 360. The fluid can be heated or cooled to a temperature between 4° C. and 42° C. (39.2° F. and 107.6° F.), and can circulate through fluid containing layer 350, exchanging thermal energy 330 with patient's skin 320 via hydrogel layer 340, so as to warm or cool patient P to the target temperature. Although in this example, thermal energy 330 is shown flowing from skin 320 to the fluid in layer 350, heat 330 can flow in either direction between patient P and layer 350, so as to heat or cool patient P to the target temperature.

Alternatively, in some embodiments, pad 120 comprises hydrogel layer 340, a thin film layer which serves as a fluid barrier, and outer insulating layer 360 comprising foam with water channels.

A hydrogel is an appropriate material for layer 340 because the hydrogel is biocompatible, its adhesive strength does not tend to increase over time as compared with traditional adhesive, it tends to envelop hair on patient's skin 320, thereby facilitating good thermal contact, and its high water content results in relatively high thermal conductivity. Accordingly, hydrogel layer 340 may function as a thermally conductive layer, while also having sufficient adhesive properties so as to integrally provide an adhesive surface. Alternatively, in some embodiments, the conformable, thermally conductive layer and adhesive surface can be comprised of different materials. For example, an appropriate adhesive material may be sprayed or otherwise applied onto the surface of a layer of an appropriate conformable, thermally conductive material different than the adhesive material.

Fluid containing layer 350 can include tortuous fluid flow paths, which can be defined by dimples or other elongated members on insulation layer 360 or within the fluid containing layer 350. Such tortuous fluid flow paths can serve to regulate the fluid flow, and to inhibit the formation of boundary layers wherein some of the fluid remains substantially stationary along the inside surfaces of the fluid containing layer 350. Such boundary layers could reduce the efficiency of the pad 120 because the stationary fluid remains within the fluid containing layer 350, but eventually becomes ineffective at heating or cooling patient P as it approaches the existing temperature of patient P. Furthermore, the crisscrossed geometry of elongated members defining the tortuous flow paths also facilitates an even, low pressure drop between the inlet and the outlet required by a negative flow pressure circulating system.

One need that frequently arises with targeted temperature management (TTM) is for a simpler, quicker method to place the TTM pad on a patient. The method of placing pad 120 outlined in the example of FIG. 2 may be difficult for clinical practitioners to follow. It may be confusing or complex to orient the pad 120 correctly, and to wrap pad 120 about the patient's body. In some examples, improperly placing the medical pad 120 could lead to poor thermal contact between the pad and the patient, and even to sub-optimal heating or cooling the patient. In an acute case, such sub-optimal heating or cooling could lead to medical complications, particularly if the patient is in a vulnerable state, such as recovering from a stroke, medical emergency, or surgery. The embodiments of TTM medical pads and methods disclosed herein below can address these problems by providing a simple, adjustable approach to placing and wrapping the TTM medical pads about a patient.

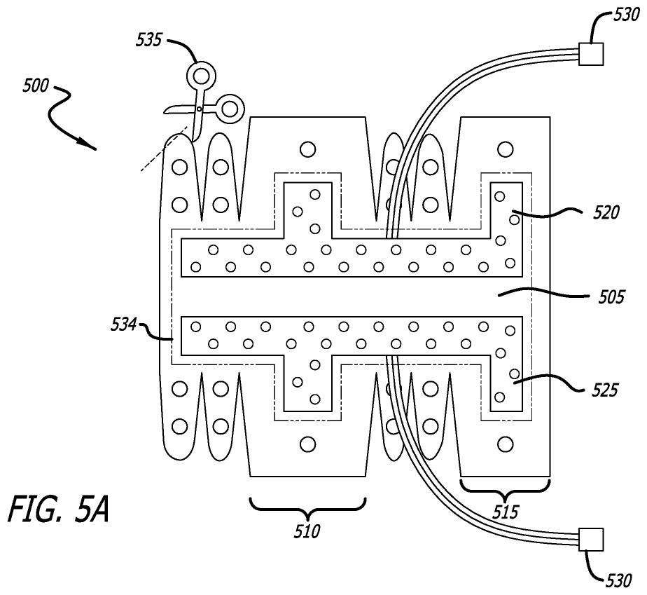

Moreover, the need for TTM medical pads to accommodate different patient sizes can cause further clinical confusion and frustration. An inadequately-fitting medical pad, such as that shown in FIG. 4, may hinder effective transmission of thermal energy between the pad and the patient, and may also give rise to patient discomfort.

FIG. 4 shows a patient P₁ with an oversized medical pad 120. In this example, as pad 120 is too large for patient P₁, portions 410 of pad 120 entirely cover the chest of patient P₁, which may run counter to the intention of the clinician overseeing the TTM therapy.

In addition to causing patient discomfort, such a situation could lead to energy waste, as well as ineffective temperature management. Because portions 410 of pad 120 overlap one another, and do not directly contact patient P₁, some of the heating and cooling power of pad 120 goes to waste. For example, since the TTM fluid flowing through pad 120 is at a substantially uniform temperature, net heat is not expected to be exchanged between the overlapping portions of pad 120. Instead, the excess flow of TTM fluid may heat or cool the ambient air around patient P₁. In another example, because portions 410 of pad 120 cover too much body surface of patient P₁, patient P₁ may be heated or cooled too strongly by pad 120. In an acute case, overheating or overcooling patient P₁ could engender a risk of medical complications, particularly if patient P₁ is in a vulnerable state, such as recovering from a stroke, from another medical emergency, or from a surgery. As a result, it can be essential that the clinician selects a properly-sized pad for patient P₁.

Typically, numerous TTM pad sizes may be available, in order to ensure a patient can be properly fitted. However, selecting the correct size may be difficult and complex. In particular, there exists a risk of selecting the wrong size pad and making multiple kit components unusable, resulting in waste of a valuable product and frustration on the part of users due to incorrect pad sizing. Moreover, maintaining numerous sizes may require a hospital or other facility to devote valuable storage space and expend staff time and effort on stocking TTM pads. Thus, there is a need for a method to adjust the size of pad 120 in order to improve its fit on patient P₁. The disclosed embodiments can also address this problem by adjusting the patient contact area to better accommodate patients of different sizes.

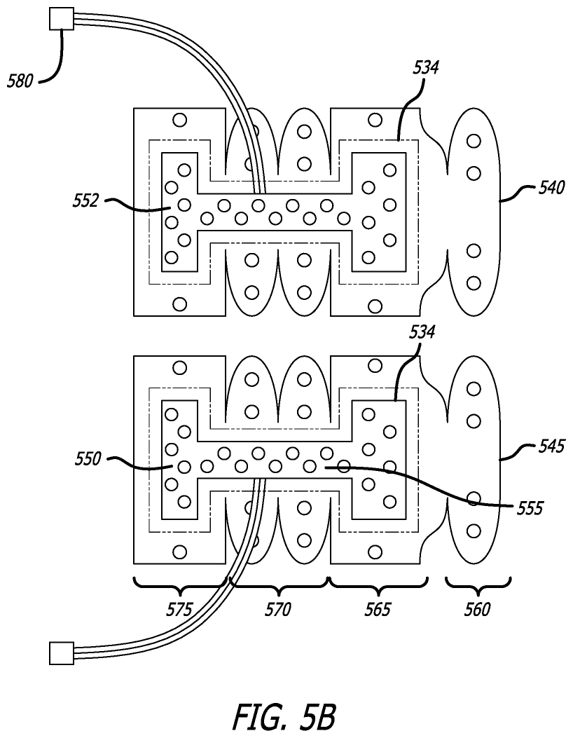

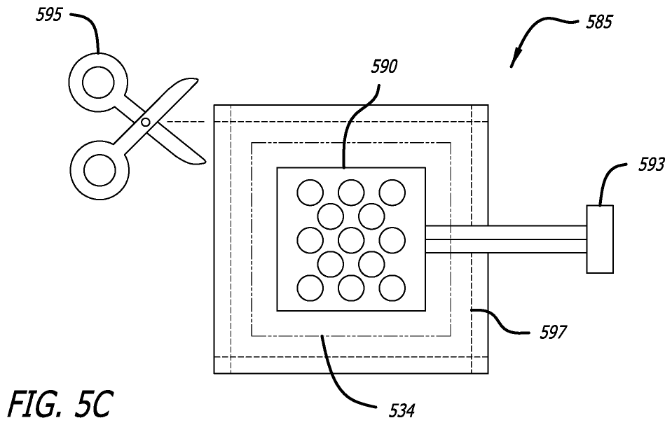

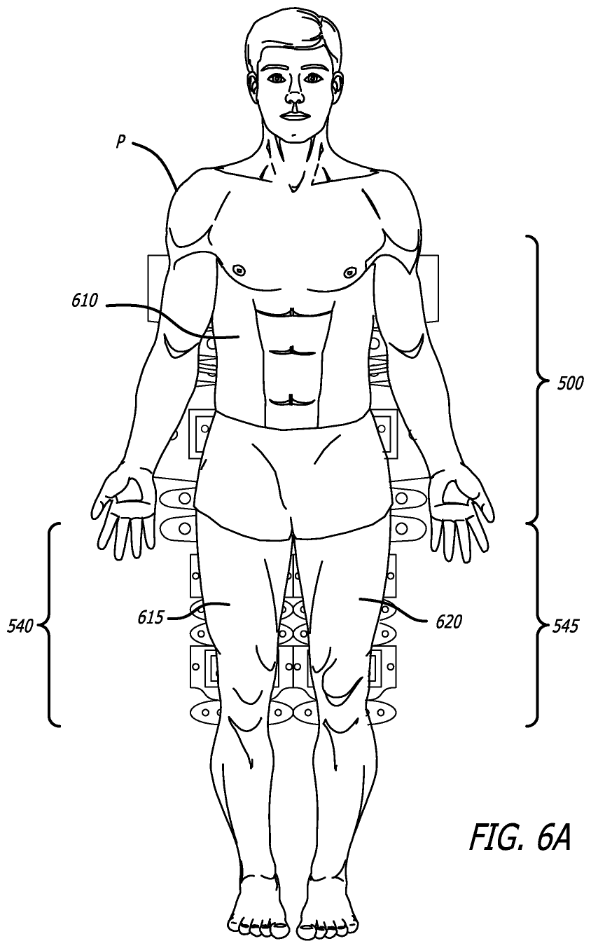

FIG. 5A illustrates an exemplary orientable and resizable medical pad 500 for a patient's torso, according to some embodiments. Pad 500 comprises a hydrogel layer 520, a fluid containing layer 525 for containing the TTM fluid, an insulation layer, inlets and outlets 530 for a fluid delivery line (FDL), a central trunk structure 505 which can align with the patient's spine, and a number of pairs of wing structures branching from the central trunk 505. In addition to its internal layers, the pad can have a patient contact surface to facilitate thermal energy exchange with the patient. The patient contact surface can be disposed over the fluid containing layer 525, for example on the central trunk structure 505.

The central trunk structure 505 may be positioned beneath the patient, e.g., the patient's back may be placed on the central trunk 505. The wings can be wrapped around to the front of a portion of the patient's torso, such as to the front of the patient's abdomen or chest. In particular, the wings can include wings 510, which can wrap around to the front of the patient's abdomen, and wings 515, which can wrap around to the front of the patient's chest. The wings may be arranged in pairs about the central trunk 505, for example wings 510 and 515 each comprise a pair of wings positioned on opposite sides of central trunk 505. The respective wings in a pair of wings can be connected to each other by a strap.

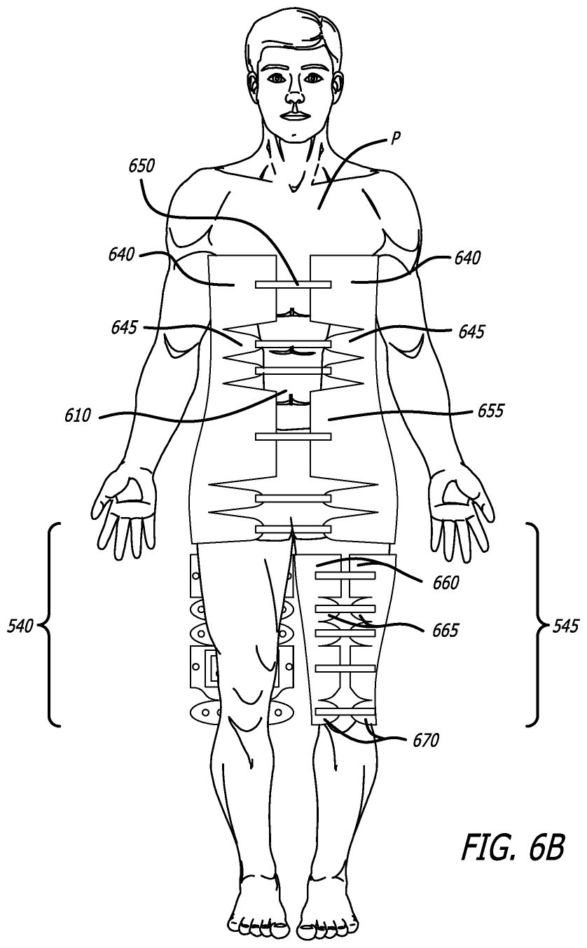

While applying pad 500, a clinician may position the central trunk structure 505 beneath the patient's spine. The patient can lie on central trunk 505 of pad 500 (see FIG. 6A). Because the patient's entire torso can be covered by a single pad 500, pad 500 is straightforward to orient correctly while being placed on the patient. In particular, unlike with conventional TTM pads, the clinician does not need to turn the patient on his or her side while placing the pads, as in the example of FIG. 2. Moreover, the central trunk 505 aligns with the patient's spine, so orienting the pad 500 is straightforward, similar to dressing the patient in a jacket or other clothing, rather than being a complex or confusing task. The clinician can then wrap the wings, such as wings 510 and 515, around to the front of a body part of the patient's, such as the patient's abdomen and chest, respectively. In a typical example, placing the pads on a patient only involves the steps of placing the pads on a bed, positioning the patient over the pads, and wrapping the pads around the patient. Because placing the disclosed pad 500 is simple, unambiguous, and straightforward, the risk of improperly placing the TTM pad, sub-optimally heating or cooling the patient, and possibly causing medical complications, is reduced.

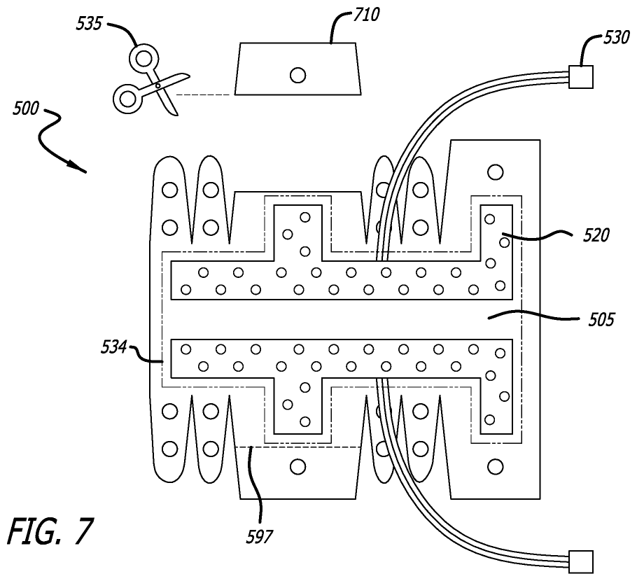

In addition, during the process of fitting or placing pad 500, the clinician can cut or trim the wings or another part of pad 500, thereby reducing the size of pad 500, as described in the example of FIG. 7 below. For example, the clinician can cut or trim pad 500 using scissors 535, as shown. In other embodiments, the pad 500 may include various tear-lines that include pre-formed perforations that extend from one side of a portion of the pad 500 to a second opposing size of the pad 500 (e.g., tear-lines may be formed in any of the wings). Thus, the discussions throughout that refer to trimming via the use of scissors also apply to trimming by tearing along tear-lines. The disclosed pad's ability to be resized easily provides advantages, such as enabling one TTM pad 500 to be used for patients of a range of sizes. As discussed herein, the wings may be trimmed in sections that do not include a fluid containing layer.

FIG. 5A also illustrates that the pad 500 may include markings, lines, annotations or other indicators (generally “markings”) 534 that indicate to a clinician an area that should not be cut or trimmed, which would typically be the fluid containing layer 350 as seen in FIG. 3. Thus, the markings 534 provide a visual warning to a clinician as to an area that should not be cut or trimmed in order to avoid leakage or otherwise damage the pad 500 (at times such damage may be irreparable). The markings 534 may be provided in various forms such as a color distinct from that of the pad 500 (e.g., the pad 500 may be a first color, blue, and the markings 534 may be a second color, red). The markings 534 may include a raised notch of material so that the clinician may obtain a visual and physical understanding of a border between the section of the pad 500 that may be cut or trimmed and the section that should not. In some instances, the markings 534 may coincide with an additional amount of material thereby making a trimming or cutting at this point more difficult than the trimming or cutting of the section of the pad 500 that is external to the markings 534 (e.g., closer to the outer edge). In some instances, the raised notched serves as the additional amount of material. In yet other embodiments, the markings 534 include text indicating the markings 534 serve as a border between a section that may be trimmed or cut and a section that should not (e.g., sample text may state “DO NOT CUT/TRIM”). Additionally, in some embodiments, the text may be accompanied by an arrow pointing inward (e.g., toward a middle of the pad 500, or otherwise away from an outer edge). In some embodiments, the markings 534 may be reflective, e.g., material formed of retroreflective lenses bonded with a special polymer layer such as 3M™ reflective materials.

FIG. 5B illustrates exemplary easily orientable and resizable medical pads 540 and 545 for a patient's legs, according to some embodiments. Pads 540 and 545 comprise a fluid containing layer 550, a hydrogel layer 552, an insulation layer, inlets and outlets 580 for a fluid delivery line (FDL), a central trunk structure 555 for contacting the patient's spine, and several pairs of wing structures branching from the central trunk. In addition, pads 540 and 545 have a patient contact surface. The patient contact surface may be disposed over the fluid containing layer 550, for example on the central trunk structure 555, and/or on any other part of the pads.

The central trunk structure 555 may be positioned beneath the patient's leg, and the wings can be wrapped around to the front of the patient's leg. In particular, the wings can include wings 560, which can wrap around to the front of the patient's waist, wings 565, which can wrap around to the front of the patient's thigh, wings 570, which can wrap around to the front of the patient's knee, and wings 575, which can wrap around to the front of the patient's calf In the case of wings 560, the wings can wrap around a portion of the patient's waist corresponding to the leg covered by the pad. For example, if pad 545 is placed on the patient's left leg, wings 560 on pad 545 can be wrapped around the left portion of the patient's waist. The wings may be arranged in pairs about the central trunk 555, for example wings 560, 565, 570, and 575 each comprise a pair of wings positioned on opposite sides of central trunk 555.

While applying pads 540 and 545, a clinician may position the central trunk structure 555 beneath the patient's legs. Because each of the patient's legs can be covered by a single pad, the pads are straightforward to orient correctly while placing on the patient. In particular, unlike with conventional TTM pads, the clinician does not need to turn the patient on his or her side while placing the pads, as in the example of FIG. 2. Moreover, the central trunk 555 aligns with the patient's leg, so orienting pads 540 and 545 is straightforward, rather than being a difficult or confusing task. The clinician can then wrap the wings, such as wings 560, 565, 570, and 575, around to the front of a body part of the patient's, such as the patient's waist, thighs, knees, and calves, respectively. Thus, in a typical example, placing the pads on a patient only involves the steps of placing the pads on a bed, positioning the patient over the pads, and wrapping the pads around the patient. Because placing the disclosed pads 540 and 545 is straightforward and unambiguous, the risks of improper placement, sub-optimal heating or cooling, and medical complications are reduced.

As in the case of pad 500, the clinician can cut or trim the wings or another part of pads 540 and 545, thereby reducing the size of the pads. As discussed herein, the wings may be trimmed in sections that do not include a fluid containing layer.

FIG. 5C illustrates an exemplary resizable universal medical pad 585, according to some embodiments. Universal pad 585 can be used as needed to provide additional coverage on an arbitrary part of a patient's body, and unlike torso and leg pads 500, 540, and 545, universal pad 585 is not designed to wrap around a specific body part. Universal pad 585 can include a fluid containing layer 590, a hydrogel layer, an insulation layer, and inlets and outlets 593 for an FDL.

Universal pad 585 can be trimmed, for example using scissors 595, as shown, thereby reducing the size of pad 585 (also see FIG. 7). The ability of pad 585 to be resized provides advantages, such as enabling universal TTM pad 585 to be adjusted to a patient's size. In some examples, universal pad 585 may be trimmed in a section that does not include a fluid containing layer, such that the patient contact area of pad 585 is not reduced. Additionally, as an alternative embodiment to the utilization of scissors, some pads, such as the universal pad 585 may include one or more tear-lines, such as the tear-lines 597. In some embodiments, the tear-lines 597 may be included in combination with the markings 534 as discussed above.

FIGS. 6A-6B illustrate placement of an exemplary easily orientable and resizable medical pad on a patient P, according to some embodiments. In this example, patient P lies with the patient's torso 610 resting on pad 500 and the patient's legs 615 and 620 resting on pads 540 and 545, respectively.

In some embodiments, the central trunk structure of pad 500 is configured to be positioned beneath the patient's torso 610, as shown in FIG. 6A. Likewise, the central trunk of pads 540 and 545 is positioned beneath the patient's legs 615 and 620. Then, while placing the pads, a clinician can wrap each pad's wings around to a front of a portion of the patient's body, as shown in FIG. 6B. For example, the wings 640 can wrap around the patient's abdomen, the wings 655 can wrap around the patient's chest, and the wings 645 can wrap around a midsection of the patient's torso 610, as shown. Likewise, the wings 660, 665, and 670 on pad 545 can wrap around the patient's thighs, knees, and calves, respectively.

Accordingly, in some examples, orienting and placing the pads 500, 540, and 545 on patient P may be faster and easier than with conventional TTM pads, particularly because just one pad 500 is needed to cover the patient's torso. The central trunks of the respective pads align with major axes of the patient's body, such as the patient's spine or legs, so orienting the disclosed pads is straightforward, similar to dressing the patient in a jacket or other clothing. Because placing the disclosed pads is simple, unambiguous, and straightforward, the risks of improperly placing the TTM pads, sub-optimally heating or cooling the patient, and possibly causing medical complications, are reduced.

The hydrogel layer and/or the patient contact surface of the TTM pads can extend onto the wings, so that some or all of the wings can make contact with the patient's skin, thereby heating or cooling patient P. Several pads can be placed so as to cover several body parts of the patient P, such as the patient's legs and torso, thereby providing sufficient thermal contact to heat or cool the patient to the desired body temperature. This thermal contact may be made through each pad's patient contact surface, which may be located on the respective pad's central trunk structure, and/or on other parts of the respective pad, such as its wings. In some examples, one or more additional universal TTM pads, such as universal pad 585, may be used to cover additional area of the patient's body. In some embodiments, the wings can be adjusted to fit patient P better, e.g., by being folded, tightened, or stretched. In some embodiments, the wings can be cut to a desired size, as disclosed herein below.

In some embodiments, some or all of the pairs of wings are configured to be fastened together via a strap, such as strap 650 connecting pair of wings 640. Such a strap can secure the wings in place and improve the pad's fit, thereby also improving patient comfort and the pad's efficiency of thermal transfer. In some embodiments, strap 650 is connected to wings 640 via hook and loop fasteners (e.g., VELCRO®). In some embodiments, the strap is connected to wings 640 via a snap, button, zipper, or clasp. In some embodiments, strap 650 is sewn, attached, or permanently fixed to one of the wings. In some embodiments, the strap 650 is adjustable, for example the length of strap 650 can be adjusted in order to tighten or loosen pad 500.

FIG. 7 illustrates trimming of an exemplary orientable and resizable medical pad 500, according to some embodiments. In this example, portion 710 is trimmed from pad 500, thereby reducing the size of pad 500. Pad 500 can be cut or trimmed, for example using scissors 535, or a similar cutting instrument. Removing portion 710 enables a clinical user to resize pad 500 as needed, for example during the pad placement process. In another example, the user can resize the TTM pad after measuring a patient but before placing the pad on the patient.

Resizing pad 500 can improve patient fit and facilitate thermal exchange, while simplifying the process of selecting the correct size and placing the pad. In particular, resizing pad 500 reduces the number of sizes a facility must purchase and maintain, as well as reducing clinician confusion and errors.

In various embodiments, the fluid containing layer may be located only within the central trunk structure 505, or may extend beyond the central trunk 505 and into the wing structures. Because the TTM fluid flows in the fluid containing layer, the pad's patient-contacting surface, which facilitates thermal energy exchange between the TTM fluid and patient, may be the portion of the pad's surface situated above the fluid containing layer.

In the case that the fluid containing layer does not extend into the trimmed portion 710 of the pad, the fluid containing layer is not trimmed. In this case, trimming the medical pad does not change the area of the patient contact surface. TTM pad 500 may also include a hydrogel layer, which may extend beyond the central trunk 505 and into the wings, including into trimmed portion 710. Thus, in some embodiments, the extension of the hydrogel layer in portion 710 is cut, even if the fluid containing layer does not extend into portion 710.

The ability to trim and resize pad 500, as illustrated in this example, provides a number of advantages. First, trimming pad 500 enables the same TTM pad 500 to be used on patients of a range of sizes. As a result, a hospital or other facility can purchase and maintain fewer sizes of TTM pads, but can still access pads capable of fitting patients properly when needed. Likewise, the disclosed resizable TTM pad can simplify a clinician's process of selecting an appropriate pad size for a given patient. In particular, since fewer sizes of TTM pads are needed, the disclosed pads and methods make selecting the correct size of TTM pad to fit a patient simpler, faster, and less confusing. In addition, the ability to resize pad 500 reduces the likelihood a clinician will select a pad of the wrong size, which could make multiple kit components unusable. Thus, the disclosed TTM pad can also reduce the risks of waste, clinician frustration, and medical complications due to incorrect pad sizing. As also illustrated, the pad 500 may include one or more tear-line 957 as discussed above.

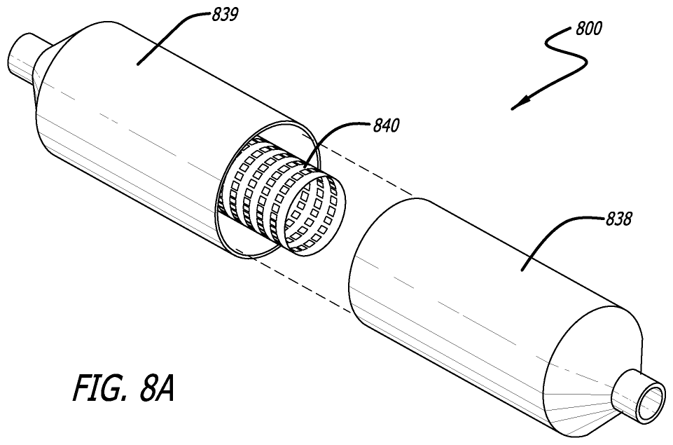

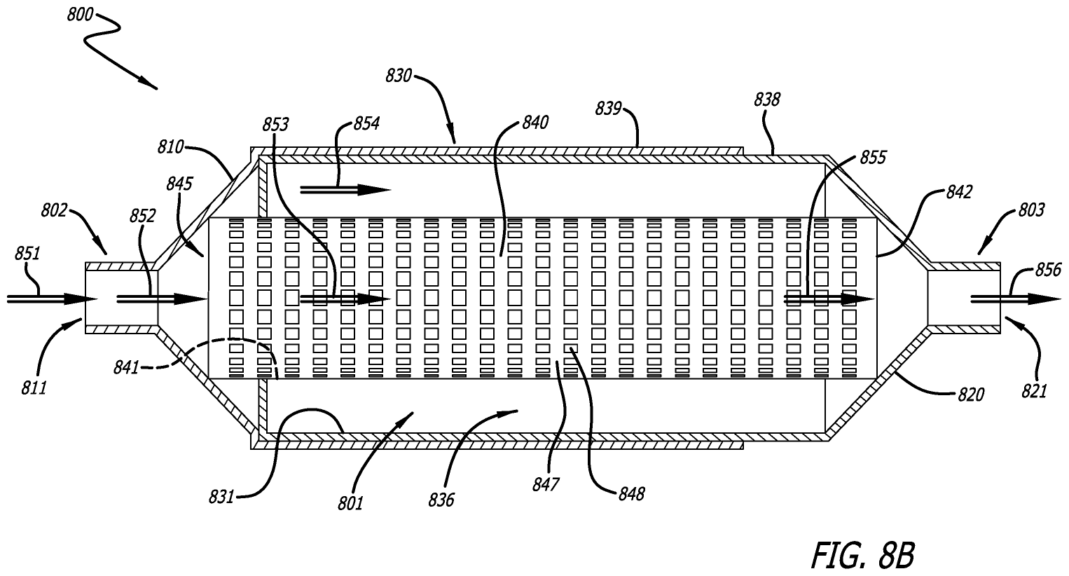

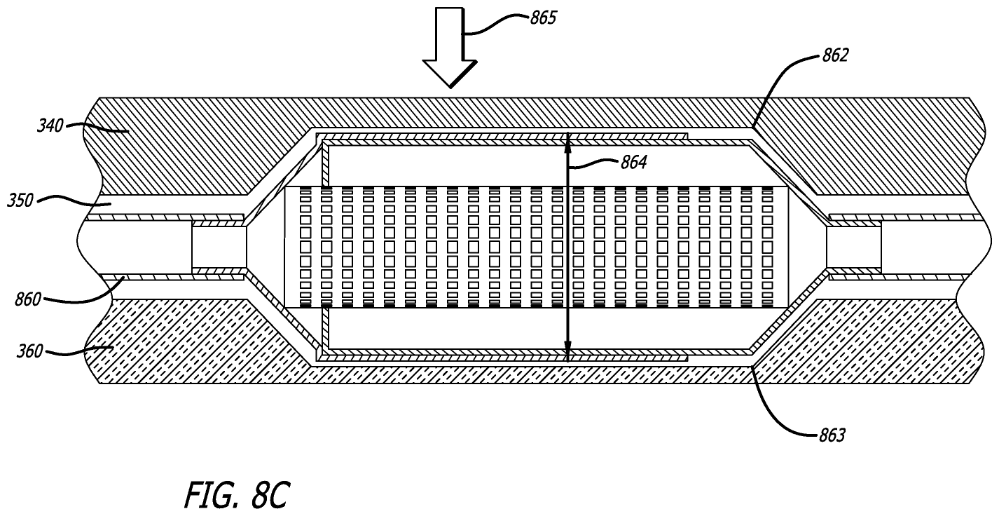

Referring now to FIGS. 8A-8B, a filter 800 is illustrated that may be included with the TTM system 100, in accordance with some embodiments. The filter 800 may be disposed in line with a TTM fluid flow path of the TTM system 100 so that the circulating TTM fluid flows through the filter 800. The filter 800 may be configured to remove (i.e., filter out) material/particles having a size of 0.2 microns or larger from the TTM fluid without causing a flow restriction of the fluid.

The filter 800 comprises a longitudinal shape having a flow path 801 extending from a first end 802 to a second end 803. The filter 800 comprises a diffuser 810 adjacent the first end 802, a nozzle adjacent 820 the second end 803, and a body 830 extending between the diffuser 810 and the nozzle 820. Along the diffuser 810, a cross-sectional flow area of the filter 800 expands from an inlet flow area 811 to a body flow area 831 and along the nozzle 820, the cross-sectional flow area of the filter 800 contracts from the body flow area 831 to an outlet flow area 821. In some embodiments, the inlet flow area 811 and the outlet flow area 821 may be substantially equal.

In some embodiments, the body flow area 831 may be constant along the body 830. In other embodiments, the body flow area 831 may vary along a length of the body 830 such that the body flow area 831 is greater or less along middle portion of the body 830 than at the ends of the body 830. In some embodiments, the body flow area 831 may be circular.

The filter 800 comprises an inner tube 840 disposed within the body 830 extending along the length of body 830. The inner tube 840 may be coupled to the diffuser 810 at a first inner tube end 841 so that fluid entering the filter 800 at the first end 802 also enters the inner tube 840 at the first inner tube end 841. The inner tube 840 may be coupled to the nozzle 820 at a second inner tube end 842 so that fluid exiting the filter 800 at the second end 803 also exits the inner tube 840 at the second inner tube end 842.

The inner tube 840 comprises an inner tube flow area 845 extending the length of the inner tube 840. The inner tube flow area 845 may be greater than the inlet flow area 811 and/or the outlet flow area 821. The inner tube flow area 845 may be constant along the length of the inner tube 840. In some embodiments, the inner tube flow area 845 may vary along the length of the inner tube 840. In some embodiments, the inner tube 840 may comprise a circular cross section. The inner tube 840 and the body 830 may be configured so that the body flow area 831 comprises a combination of the inner tube flow area 845 and an annular flow area 836.

The inner tube 840 comprises a porous a circumferential wall 847. The porous wall 847 may be configured so that fluid may flow through the porous wall 847, i.e., through the pores 848 of the porous wall 847. Consequently, fluid may flow through the porous wall 847 from the inner tube flow area 845 to the annular flow area 836 and from the annular flow area 836 into the inner tube flow area 845.

In use, the longitudinal velocity of the fluid may change along the length of the filter 800. As the volumetric fluid flow through the filter is constant, the longitudinal velocity of the fluid may be at least partially defined by the flow areas of the filter 800 as described below. The fluid may enter the filter 800 at a first longitudinal velocity 851 and decrease along the diffuser so that the fluid enters the inner tube at a second velocity 852 less than the first longitudinal velocity 851. At this point, a portion of the fluid may flow through the porous wall 847 from the inner tube flow area 845 into the annular flow area 836 to divide the fluid flow into a third velocity 853 within the inner tube flow area 845 and a fourth velocity 854 within the annular flow area 836. The fourth velocity 854 may be less than the third velocity 853. A portion of the fluid may then flow back into the inner tube flow area 845 from the annular flow area 836 to define a fifth velocity 855 along the inner tube flow area 845 which may be about equal to the second velocity 852. The fluid may then proceed along the nozzle 820 to define a sixth velocity 856 exiting the filter 800. In some embodiments, the first velocity 851 and the sixth velocity 856 may be about equal.

The filter 800 may be configured to remove harmful bacteria and viruses from the fluid using sedimentation principles. In use, the filter 800 may be oriented horizontally so that the direction of fluid flow through the filter 800 is perpendicular to a gravitational force 865. In some instances, bacteria, viruses, and other particles within the fluid may have a greater density than the fluid and as such may be urged by the gravitational force 865 (i.e., sink) in a direction perpendicular to the fluid flow direction. In some instances, particles within the inner tube flow area 845 may sink toward and through the porous wall 847 into the annular flow area 836. Particles within the annular flow area 836 may then sink toward an inside surface 831 of the body 830 and become trapped adjacent the inside surface 831. The geometry of the filter 800 may be configured to allow 0.2-micron bacteria/virus particles to fall out of the flow of TTM fluid and become trapped along the inside surface 831.

In some embodiments, the filter 800 may be configured so that flow of fluid from the inner tube flow area 845 into the annual flow area 836 my drag particles through the porous wall 847. In some embodiments, the inlet flow area 811, the inner tube flow area 845, and the annual flow area 836 may be sized so that the third velocity 853 is less than about 50 percent, 25 percent, or 10 percent of the first velocity 851 or less. In some embodiments, the body 830 and the inner tube 840 may be configured so that the fourth velocity 854 is less than the third velocity 853. In some embodiments, the fourth velocity 854 may less than about 50 percent, 25 percent, or 10 percent of the third velocity 853 or less.

In some embodiments, the filter 800 may be configured so that the flow within the inner tube flow area 845 is laminar flow, i.e., so that the velocity of the fluid flow adjacent to or in close proximity to an inside surface 841 of the porous wall 847 is less than the velocity at a location spaced away from the inside surface 841. In such an embodiment, the particles may more readily sink toward and through the porous wall 847.

In some embodiments, the filter 800 may be configured so that the fluid flow within the annual flow area 836 is laminar flow, i.e., so that the velocity of the fluid flow adjacent to or in close proximity to inside surface 831 of the body 830 is less than the velocity at a location spaced away from the inside surface 831. In such an embodiment, the particles may more readily sink toward and be trapped along the inside surface 831.

The filter 800 may comprise three components including the inner tube 840 an inner body shell 838, and an outer body shell 839. Each of the three components may be formed via the plastic injection molding process. Assembly of the filter 800 may include capturing the inner tube 840 within the inner body shell 838 and the outer body shell 839 and sliding the inner body shell 838 into the outer body shell 839 wherein the fit between the inner body shell 838 and the outer body shell 839 is an interference fit.

In some embodiments, the filter 800 may be disposed within the pad 120. FIG. 8C shows a detail cross-sectional view of the pad 120 including the filter 800 disposed within the fluid containing layer 350. The filter 800 is coupled in line with an internal flow path 860 within the fluid containing layer 350 so that fluid circulating within the pad 120 passes through the filter 800. The filter 800 may be sized so that the inlet flow area 811 and the outlet flow area 821 are similar to a cross-sectional flow area of the internal flow path 860 within the fluid containing layer 350. The internal flow path 860 may be comprised of tubing (e.g., similar to the fluid delivery lines 140) that is disposed within the fluid containing layer 350. In such embodiments, the tubing of the internal flow path 860 receives the fluid from a fluid delivery line 140 at an inlet port. The fluid flows through the tubing of the internal flow path 860, passing through the filter 800, toward an outlet port, at which point the fluid exits the pad 120 and is received by a second fluid delivery line 140.

In some embodiments, a thickness of the fluid containing layer 350 may increase adjacent the filter 800 to accommodate a body diameter 864 of the filter 800. To further accommodate the body diameter 864, the insulation layer 360 and/or the thermal conduction layer 340 may comprise internal depressions 862, 863, respectively.

In some embodiments, one or more filters 800 may be disposed in line with the flow of fluid at other locations of the TTM system 100. In some embodiments, one or more filters 800 may be disposed within the TTM module 110. In some embodiments, one or more filters 800 may be disposed in line with one or more of the fluid delivery lines 140. In some embodiments, the filter 800 may be disposed in line with a fluid conduit of the pad external to the fluid containing layer 350 such as a conduit extending between a pad connector and the pad 120.

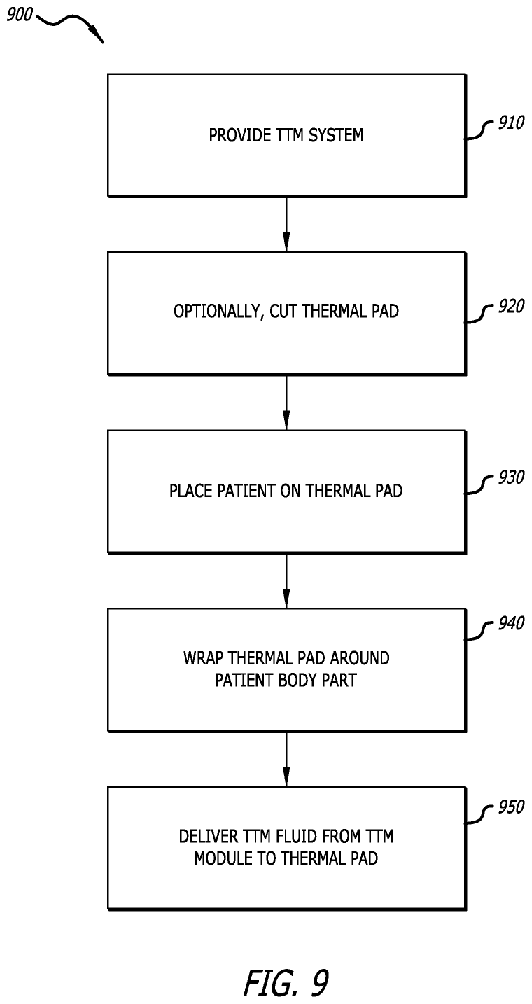

FIG. 9 illustrates a flowchart of a method 900 for using an easily orientable and resizable TTM medical pad, according to some embodiments. Each block illustrated in FIG. 9 represents an operation performed in the method 900 of using an orientable and resizable TTM medical pad. In various embodiments, the method can be performed by one or more users, such as nurses, doctors, or other clinicians, etc.

As an initial step in the method 900, the user can provide a TTM system (block 910) comprising a TTM module configured to provide a TTM fluid, a thermal pad configured to receive the TTM fluid from the TTM module to facilitate thermal energy transfer between the TTM fluid and a patient, and a fluid delivery line (FDL) extending between the TTM module and the thermal pad.

The pad comprises a fluid containing layer for containing and circulating the TTM fluid, a patient contact surface to facilitate thermal energy exchange with the patient, a central trunk structure, and at least two wings branching from the central trunk structure. The wings may be arranged in pairs about the central trunk.

In addition to its internal layers, the pad can have a patient contact surface. The patient contact surface can facilitate thermal energy exchange with the patient, that is, thermal energy can flow between the TTM fluid and the patient's skin through the patient contact surface. Accordingly, the patient contact surface can be disposed over the fluid containing layer, for example on the central trunk structure.

Next, the user can optionally cut the thermal pad (block 920). For example, the user can cut or trim the thermal pad using scissors, or a similar cutting instrument. In a typical example, the user can cut a portion off of one or more wings of the thermal pad. Alternatively, the user can cut any other portion or section of the pad, and is not limited by the present disclosure. Cutting the thermal pad can reduce the size of the pad. The ability to resize the pad provides advantages, such as enabling the pad to be adjusted to a patient's size, and reducing the number of different pad sizes that must be maintained in stock.

In preparation for placing the pads on the patient, the user can also place the one or more thermal pads on a bed.

Next, the user can place the patient on the thermal pad (block 930). For example, if the one or more thermal pads are positioned on a bed, the clinical user can position the patient on the bed over the pads. The user can position the patient with the patient's torso resting on a torso pad, and each of the patient's legs resting on a leg pad. In some examples, the user can apply one or more additional universal pads to increase TTM coverage, for example on an uncovered portion of the patient's upper or lower body.

The central trunk structure of the torso pad may be positioned beneath the patient, e.g. the patient may lie with the patient's back on the torso pad's central trunk. Likewise, the patient may lie with each of the patient's legs on a respective leg pad's central trunk. In some examples, orienting and placing the patient on the pads may be faster and easier than with conventional TTM pads, particularly because just one pad is needed for the patient's torso. As the central trunk of each pad can align with the patient's torso or leg, respectively, orienting the pads can be straightforward, comparable to dressing the patient in a jacket or other clothing. Moreover, because placing the disclosed pad 500 is simple, unambiguous, and straightforward, the risks of improperly placing the TTM pad, sub-optimally heating or cooling the patient, and possibly causing medical complications, are reduced.

Next, the user can wrap the thermal pad around a body part of the patient (block 940). In a typical example, the user wraps each pair of wings around a portion of the patient's body. For example, on a torso pad, the wings can be wrapped around to the front of a portion of the patient's torso, such as to the front of the patient's abdomen or chest. The user can connect the wings to each other using a strap.

Finally, the user can configure the TTM system to deliver the TTM fluid from the TTM module to the thermal pad via the fluid delivery line (FDL) (block 950). The TTM fluid can circulate through the fluid containing layer of the TTM pads, thereby heating or cooling the patient.

While some particular embodiments have been disclosed herein, and while the particular embodiments have been disclosed in some detail, it is not the intention for the particular embodiments to limit the scope of the concepts provided herein. Additional adaptations and/or modifications can appear to those of ordinary skill in the art, and, in broader aspects, these adaptations and/or modifications are encompassed as well. Accordingly, departures may be made from the particular embodiments disclosed herein without departing from the scope of the concepts provided herein.

What is claimed is:

1. A medical pad for exchanging thermal energy between a targeted temperature management (TTM) fluid and a patient, the medical pad comprising:
a fluid containing layer for containing the TTM fluid, the fluid containing layer configured for circulating the TTM fluid within the fluid containing layer;
a patient contact surface to facilitate thermal energy exchange with the patient;
a central trunk structure, wherein the patient contact surface is disposed at least on part of the central trunk structure; and
at least two wings branching from the central trunk structure, wherein the at least two wings are configured to be wrapped around a portion of a body of the patient.

2. The medical pad of claim 1, wherein a respective wing of the at least two wings is further configured to be trimmed, thereby reducing a size of the medical pad.

3. The medical pad of claim 2, wherein:
the patient contact surface is substantially aligned over the fluid containing layer, thereby facilitating the thermal energy exchange with the TTM fluid circulating within the fluid containing layer,
the respective wing is configured to be trimmed in a respective edge section of the respective wing, wherein the fluid containing layer does not extend within the respective edge section, and
the reducing the size of the medical pad does not reduce an area of the patient contact surface.

4. The medical pad of claim 3, wherein:
the medical pad further comprises a conformable, thermally conductive hydrogel layer,
the hydrogel layer extends to the respective edge section of the respective wing, and
the hydrogel layer is configured to be trimmed together with the respective wing.

5. The medical pad of claim 2, wherein:
the patient contact surface is substantially aligned over the fluid containing layer, thereby facilitating the thermal energy exchange with the TTM fluid circulating within the fluid containing layer,
the patient contact surface is further disposed at least on a respective section of the respective wing, the fluid containing layer disposed at least partly within the respective section,
the respective wing is configured to be trimmed in the respective section, and
the reducing the size of the medical pad reduces an area of the patient contact surface.

6. The medical pad of claim 1, wherein:
the at least two wings comprise a plurality of pairs of wings,
each pair of wings comprises two wings on opposite sides of the central trunk structure, and
each pair of wings is configured to be wrapped around the portion of the body of the patient.

7. The medical pad of claim 1, wherein the portion of the body of the patient comprises a leg of the patient.

8. The medical pad of claim 1, wherein the portion of the body of the patient comprises a torso of the patient.

9. The medical pad of claim 1, wherein the central trunk structure is configured to be positioned beneath the patient, and the at least two wings are configured to be wrapped around to a front of the portion of the body of the patient.

10. The medical pad of claim 1, wherein the at least two wings are configured to be fastened together via a strap.

11. The medical pad of claim 10, wherein the strap is connected to the at least two wings via hook and loop fasteners.

12. The medical pad of claim 10, wherein the strap is connected to the at least two wings via a snap, button, zipper, or clasp.

13. The medical pad of claim 10, wherein the strap is sewn, attached, or fixed to a first wing of the at least two wings.

14. A universal medical pad for exchanging thermal energy between a targeted temperature management (TTM) fluid and a patient, the universal medical pad comprising:
a fluid containing layer for containing the TTM fluid, the fluid containing layer configured for circulating the TTM fluid within the fluid containing layer; and
a patient contact surface to facilitate thermal energy exchange with the patient,
wherein the universal medical pad is configured to be trimmed, thereby reducing a size of the universal medical pad.

15. A method of providing a targeted temperature management (TTM) therapy to a patient, comprising:
providing a TTM system comprising:
a TTM module configured to provide a TTM fluid;
a thermal pad configured to receive the TTM fluid from the TTM module to facilitate thermal energy transfer between the TTM fluid and a patient; and
a fluid delivery line (FDL) extending between the TTM module and the thermal pad, the FDL configured to provide TTM fluid flow between the TTM module and the thermal pad, wherein the thermal pad comprises
a patient contact surface to facilitate thermal energy exchange with the patient;
a central trunk structure, wherein the patient contact surface is disposed at least on part of the central trunk structure; and
at least two wings branching from the central trunk structure;
applying the pad to the patient, wherein applying the pad comprises wrapping the at least two wings around a portion of a body of the patient; and
delivering TTM fluid from the TTM module to the thermal pad.

16. The method of claim 15, further comprising trimming a respective wing of the at least two wings, thereby reducing a size of the thermal pad.

17. The method of claim 16, wherein:
the patient contact surface is substantially aligned over the fluid containing layer, thereby facilitating the thermal energy exchange with the TTM fluid circulating within the fluid containing layer,
trimming the respective wing further comprises cutting a respective edge section of the respective wing, wherein the fluid containing layer does not extend within the respective edge section, and
the reducing the size of the medical pad does not reduce an area of the patient contact surface.

18. The method of claim 17, wherein:
the medical pad further comprises a conformable, thermally conductive hydrogel layer,
the hydrogel layer extends to the respective edge section of the respective wing, and
trimming the respective wing further comprises trimming the hydrogel layer together with the respective wing.

19. The method of claim 16, wherein:
the patient contact surface is substantially aligned over the fluid containing layer, thereby facilitating the thermal energy exchange with the TTM fluid circulating within the fluid containing layer,
the patient contact surface is further disposed at least on a respective section of the respective wing, the fluid containing layer disposed at least partly within the respective section,
trimming the respective wing further comprises cutting the respective section, and
the reducing the size of the medical pad reduces an area of the patient contact surface.

20. The method of claim 15, wherein:
the at least two wings comprise a plurality of pairs of wings,
each pair of wings comprises two wings on opposite sides of the central trunk structure, and
wrapping the at least two wings around a portion of a body of the patient further comprises wrapping each pair of wings around the portion of the body of the patient.

21. The method of claim 15, wherein wrapping the at least two wings around the portion of the body of the patient comprises wrapping the at least two wings around a leg of the patient.

22. The method of claim 15, wherein wrapping the at least two wings around the portion of the body of the patient comprises wrapping the at least two wings around a torso of the patient.

23. The method of claim 15, wherein:
applying the pad to the patient further comprises positioning the central trunk beneath the patient, and
wrapping the at least two wings further comprises wrapping the at least two wings around to a front of the portion of the body of the patient.

24. The method of claim 15, further comprising fastening the at least two wings together via a strap.

25. The method of claim 24, further comprising connecting the strap to the at least two wings via hook and loop fasteners.

26. The method of claim 24, further comprising connecting the strap to the at least two wings via a snap, button, zipper, or clasp.

27. The method of claim 24, wherein the strap is sewn, attached, or fixed to a first wing of the at least two wings.
